(19) United States
(12) Patent Application Publication
(10) Pub. No.: US 2025/0345340 A1
(43) Pub. Date: Nov. 13, 2025

(54) METHODS FOR THE PREVENTION AND TREATEMENT OF POST-OPERATIVE ABDOMINAL ADHESIONS

(71) Applicant: UNIVERSITY OF ROCHESTER, Rochester, NY (US)

(72) Inventors: Alayna LOISELLE, Rochester, NY (US); Nicole A. WILSON, Rochester, NY (US); Matt KOTTMANN, Rochester, NY (US)

(73) Assignee: UNIVERSITY OF ROCHESTER, Rochester, NY (US)

(21) Appl. No.: 19/066,753

(22) Filed: Feb. 28, 2025

Related U.S. Application Data
(60) Provisional application No. 63/646,086, filed on May 13, 2024.

(51) Int. Cl. A61K 31/53; A61P 17/02

(52) U.S. Cl. CPC A61K 31/53; A61P 17/02

(57) ABSTRACT

Methods for prevention of post-surgical abdominal adhesions, or treatment of post-surgical abdominal adhesions by administration of ogerin, a small molecule that activates GPR68, blunting TGF-β, which halts myofibroblast differentiation. The ogerin may be administered via intra-peritoneal injection or direct application to the peritoneal cavity. The ogerin is administered to the subject as an effective amount of ogerin prior to an abdominal surgery or within 48 hours of the abdominal surgery.

This application claims priority from U.S. Provisional App. No. 63/646,086, filed May 13, 2024, which is incorporated herein by reference.

### FIELD

This application relates generally to the field of medical treatment, and in particular, to the prevention of the development, or treatment of, post-operative abdominal adhesions.

### BACKGROUND

Abdominal adhesions occur in response to peritoneal tissue trauma during abdominal and pelvic surgeries. Studies suggest that adhesions form after 67-93% of abdominal surgeries, are nearly universal in patients who have undergone multiple abdominal operations, and are the most frequent complication of abdominopelvic surgery. Serious consequences resulting from postoperative abdominal adhesions include intestinal obstruction (49-74% incidence), female infertility and dyspareunia (15-20% incidence), and chronic pain (20-50% incidence). In addition, the need for adhesiolysis (the removal or lysis of abdominal adhesions) during subsequent surgeries carries an increased risk of visceral injury, infectious complications, longer hospital stays, and increased operative time and difficulty. In the US alone, the total cost of hospital and surgical expenditures for the management of adhesion-related complications has increased from $1.3 billion in 1994 to $5 billion in 2001 and continues to increase as the number of adhesiolysis procedures has increased in the past two decades. Thus, the prevention and/or amelioration of abdominal adhesions has the potential to save billions of dollars and improve hundreds of thousands of lives.

Current treatments for abdominal adhesions include laparoscopic or open adhesiolysis. Regardless of surgical technique, adhesions form and tend to reform even after adhesiolysis procedures. As a result, efforts have been made towards developing solid or liquid physical barriers that cover injured peritoneal surfaces until re-epithelialization is complete. However, while these physical barriers may minimize postoperative adhesion formation, they have demonstrated no significant improvements in clinical outcomes due to adhesion-related complications (e.g., reoperation rates, chronic abdominal pain, or infertility). Furthermore, the use of physical barrier products has been associated with increased risk of intraabdominal infections, anastomotic leak, and higher cost. Despite promising results in animal studies, new systemic agents have not yet shown similar efficiency in humans. Therefore, while the clinical burden of postoperative abdominal adhesions is astonishingly high, there are no currently effective treatments for prevention and/or amelioration of abdominal adhesions.

### SUMMARY

One aspect of the application is a method of preventing or reducing development of post-surgical abdominal adhesions in a subject, comprising the step of administering to the subject an effective amount of ogerin prior to an abdominal or pelvic surgery or within 48 hours of the abdominal or pelvic surgery.

An aspect of the present application relates a method of treating post-surgical abdominal adhesion in a subject, comprising the step of administering to a subject in need of such treatment an effective amount of ogerin, wherein the subject has undergone abdominal or pelvic surgery and is at risk of post-surgical abdominal adhesion formation.

### BRIEF DESCRIPTION OF THE DRAWINGS

FIG. 1 shows the protocol for the experiments conducted herein.

FIG. 2 shows images demonstrating that ogerin treatment blunts adhesion formation in a mouse model. Panel A: vehicle. Panel B: ogerin-treated.

FIG. 3 shows clinical scoring of adhesion severity.

FIG. 4 shows that ogerin treatment reduces clinical adhesion score.

FIG. 5 shows effects of ogerin treatment on adhesion length.

FIG. 6 shows that ogerin reduces total adhesion burden as measured by area.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a”, “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to the value”, “greater than or equal to the value” and possible ranges between values are also disclosed, as appropriately understood by the skilled artisan.

### I. Definitions

The term “Ogerin composition” and “pharmaceutical composition” are used herein with reference to a composition comprising Ogerin and at least one pharmaceutically acceptable carrier. When referring to these compositions with regard to dosages, the weights are given in terms of the amount by weight of Ogerin.

The term “subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a disease or condition.

A “subject in need thereof means a subject identified as in need of a therapy or treatment.

A “therapeutic effect” relieves, to some extent, one or more of the symptoms of a disease or disorder. “Curing” means that the symptoms of active disease are eliminated. However, certain long-term or permanent effects of the disease may exist even after a cure is obtained (such as tissue damage and the like).

The phrase “therapeutically effective amount” as used herein refers to an amount of Ogerin that ameliorates, attenuates, or eliminates one or more of the symptoms of a particular disease or condition or prevents, modifies, or delays the onset of one or more of the symptoms of a disease or condition.

“Treat”, “treatment,” and “treating,” as used herein, refer to administering an Ogerin composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a condition or disease for a period, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Administering” means providing a pharmaceutical agent or composition to a subject, and includes, but is not limited to, administering by a medical professional and self-administering.

Administration of the ogerin compositions of the present application can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. One of ordinary skill in the art will understand that the form of administration of ogerin is not limiting, and may include, but is not limited to, powder, cream, lotion, as well as liquid forms used for injection at the site or any other appropriate location on the body of a subject.

“Parenteral administration,” means administration through injection or infusion. Parenteral administration includes, but is not limited to, subcutaneous administration, intravenous administration, intramuscular administration, intraarterial administration, and intracranial administration.

“Intraperitoneal administration” means administration into the peritoneal cavity.

“Subcutaneous administration” means administration just below the skin.

“Intravenous administration” means administration into a vein.

“Intraarterial administration” means administration into an artery.

“Pharmaceutical composition” means a mixture of substances suitable for administering to an individual that includes a pharmaceutical agent. For example, a pharmaceutical composition may comprise a modified oligonucleotide and a sterile aqueous solution.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable carrier” or “pharmaceutically acceptable excipient” includes all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form, however, does not imply that the dosage form is administered once per day or once per course of therapy.

As used herein, the term “effective amount” is an amount necessary or sufficient to achieve the desired biological effect or the selected result, and such amount can be determined by one of ordinary skill in the art as a matter of routine experimentation.

The term ‘comprising’ encompasses ‘including’ as well as ‘consisting’, e.g. a composition ‘comprising X’ may consist exclusively of X or may include something additional, e.g. X+Y.

The term ‘about’ in relation to a numerical value x is optional and means, e.g. x±10%.

The word ‘substantially’ does not exclude ‘completely’, e.g. a composition which is ‘substantially free from Y’ may be completely free from Y. Where relevant, the word ‘substantially’ may be omitted from the definition of the invention.

II. Method of Preventing and/or Treating Post-Operative Abdominal Adhesions

Abdominal adhesion tissue is composed of a disorganized extracellular matrix (ECM) connecting intra-abdominal and pelvic structures, including the partial peritoneum, intra-abdominal, and intra-pelvic organs. While the initial activation of tissue resident fibroblasts is driven, in part, by macrophages, the subsequent differentiation of tissue resident fibroblasts to matrix-producing myofibroblasts is a critical driver of adhesion maturation. Moreover, even upon resolution of inflammation and injury, myofibroblasts can remain in a sub-activated state such that they are a ‘primed’ population for rapid response to subsequent insult. This failure to clear myofibroblasts or revert them to a true basal fibroblast state may underpin the propensity for adhesions to reform and the increased incidence of adhesion formation with repeated surgery.

The inventors discovered that ogerin, a positive allosteric modulator of the proton-sensing G protein coupled receptor Gpr68, can prevent the formation of post-operative abdominal adhesions in a murine model. This finding represents a novel application of ogerin.

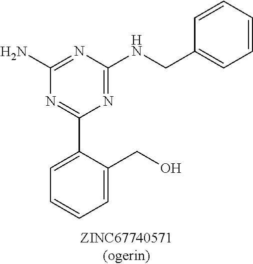 [SMILES: Nc1nc(NCc2ccccc2)nc(-c2ccccc2CO)n1]

One aspect of the present application relates to a method of preventing or reducing development of post-surgical abdominal adhesions in a subject, comprising the steps of: administering to the subject an effective amount of ogerin prior to an abdominal or pelvic surgery or within 48 hours of the abdominal or pelvic surgery. One of ordinary skill in the art will understand that the form of administration of ogerin is not limiting, and may include, but is not limited to, powder, cream, lotion, as well as liquid forms used for injection at the site or any other appropriate location on the body of a subject.

In certain embodiments, the subject is a human subject.

In certain embodiments, the ogerin is administered within 24 hours of the completion of the abdominal surgery.

In certain embodiments, the ogerin is administered within 12 hours of the completion of the abdominal surgery.

In certain embodiments, the ogerin is administered within 24, 12 or 6 hours prior to the abdominal surgery.

In certain embodiments, the ogerin is administered via intra-peritoneal injection or direct application to the peritoneal cavity.

In certain embodiments, the ogerin is administered to a human subject at a dosage in the range of 0.1-40 mg/kg, 0.1-0.3 mg/kg, 0.1-1 mg/kg, 0.1-3 mg/kg, 0.1-10 mg/kg, 0.1-20 mg/kg, 0.3-1 mg/kg, 0.3-3 mg/kg, 0.3-10 mg/kg, 0.3-20 mg/kg, 0.3-40 mg/kg, 1-3 mg/kg, 1-10 mg/kg, 1-20 mg/kg, 1-40 mg/kg, 3-10 mg/kg, 3-20 mg/kg, 3-40 mg/kg, 10-20 mg/kg, 10-40 mg/kg, or 20-40 mg/kg.

In certain embodiments, the ogerin is administered to a human subject at a dosage in the range of 5-5000 mg, 5-10 mg, 5-30 mg, 5-100, mg, 5-300 mg, 5-1000 mg, 5-2500 mg, 10-30 mg, 10-100, mg, 10-300 mg, 10-1000 mg, 10-2500 mg, 10-5000 mg, 30-100, mg, 30-300 mg, 30-1000 mg, 30-2500 mg, 30-5000 mg, 100-300 mg, 100-1000 mg, 100-2500 mg, 100-5000 mg, 300-1000 mg, 300-2500 mg, 300-5000 mg, 1000-2500 mg, 1000-5000 mg, or 2500-5000 mg.

In certain embodiments, ogerin is administered by a single administration within 24, 12 or 6 hours prior to the operation, at the time of the operation, or within 12, 24 or 48 hours of the operation. In certain embodiments, ogerin is administered daily for a period of 2, 3, 4, 5, 6 or 7 days. In certain embodiments, ogerin is administered every other day for a period of 3, 5 or 7 days.

In certain embodiments, the method further comprises the additional step of applying a physical barrier to the surgery's site, wherein the physical barrier covers peritoneal surfaces until re-epithelialization is complete.

Another aspect of the present application relates a method of treating post-surgical abdominal adhesion in a subject, comprising the step of: administering to a subject in need of such treatment an effective amount of ogerin, wherein the subject has undergone abdominal or pelvic surgery and is at risk or showing symptoms of post-surgical abdominal adhesion formation. One of ordinary skill in the art will understand that the form of administration of ogerin is not limiting, and may include, but is not limited to, powder, cream, lotion, as well as liquid forms used for injection at the site or any other appropriate location on the body of a subject.

In certain embodiments, the subject has undergone abdominal or pelvic surgery and has one or more symptoms of post-surgical abdominal adhesions.

In certain embodiments, the subject has undergone abdominal or pelvic surgery and is asymptomatic for a presence of abdominal adhesions.

In certain embodiments, the subject is a human subject.

In certain embodiments, the ogerin is administered via intra-peritoneal injection or direct application to the peritoneal cavity.

In certain embodiments, ogerin is administered to a human subject at a dosage in the range of 0.1-40 mg/kg, 0.1-0.3 mg/kg, 0.1-1 mg/kg, 0.1-3 mg/kg, 0.1-10 mg/kg, 0.1-20 mg/kg, 0.3-1 mg/kg, 0.3-3 mg/kg, 0.3-10 mg/kg, 0.3-20 mg/kg, 0.3-40 mg/kg, 1-3 mg/kg, 1-10 mg/kg, 1-20 mg/kg, 1-40 mg/kg, 3-10 mg/kg, 3-20 mg/kg, 3-40 mg/kg, 10-20 mg/kg, 10-40 mg/kg, or 20-40 mg/kg.

In certain embodiments, the ogerin is administered to a human subject at a dosage in the range of 5-5000 mg, 5-10 mg, 5-30 mg, 5-100, mg, 5-300 mg, 5-1000 mg, 5-2500 mg, 10-30 mg, 10-100, mg, 10-300 mg, 10-1000 mg, 10-2500 mg, 10-5000 mg, 30-100, mg, 30-300 mg, 30-1000 mg, 30-2500 mg, 30-5000 mg, 100-300 mg, 100-1000 mg, 100-2500 mg, 100-5000 mg, 300-1000 mg, 300-2500 mg, 300-5000 mg, 1000-2500 mg, 1000-5000 mg, or 2500-5000 mg.

In certain embodiments, ogerin is administered by a single administration. In certain embodiments, ogerin is administered daily for a period of 2, 3, 4, 5, 6, 7, 8, 9 or 10 days. In certain embodiments, ogerin is administered every other day for a period of 1, 2 or 3 weeks.

The specific dose of the ogerin treatment may be determined based on the particular circumstances of the individual patient including the size, weight, age and sex of the patient, and the route of administration of the ogerin composition.

In certain embodiments, the ogerin may be administered once, twice, three times or four times per day with the doses given at equal intervals throughout the day and night to maintain a constant presence of the ogerin compound to provide sufficient anti-adhesion activity. However, a skilled artisan will appreciate that a treatment schedule can be optimized for any given patient, and that administration of compound may occur less frequently than once per day.

In other embodiments, ogerin treatment of the present application is prescribed to be taken in combination with other anti-adhesion and/or the other active agents. When used in such combinations, the ogerin composition of the present application and other anti-adhesion agents may be administered simultaneously, by the same or different routes, or at various times during treatment.

The treatment may be carried out for as long a period as necessary, i.e., until abdominal adhesion is no longer a threat to the host. The treating physician can determine whether to increase, decrease, or interrupt treatment based on a patient's response.

### III. Pharmaceutical Compositions

Another aspect of the present invention relates to a pharmaceutical composition comprising (1) ogerin and (2) a pharmaceutically acceptable carrier, wherein the pharmaceutical composition is formulated for intraperitoneal administration or direct administration to the peritoneal cavity.

In some embodiments, the ogerin is administered in the form of a solution or suspension. Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELTM (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene, glycol, and liquid polyethylene glycol, and the like), and suitable mixtures thereof. The proper fluidity can be maintained, for example, using a coating such as lecithin, by the maintenance of the required particle size in the case of dispersion and using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating an active agent in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished using nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and poly lactic acid. Methods for preparation of such formulations will be apparent to those skilled in the art. The materials can also be obtained commercially from e.g., Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Suitable unit dosage forms include, but are not limited to powders, tablets, pills, capsules, lozenges, suppositories, patches, nasal sprays, injectables, implantable sustained-release formulations, lipid complexes, etc.

A dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the present application is dictated by and directly dependent on the unique characteristics of the active compound and the particular therapeutic effect to be achieved, and the limitations inherent in the art of compounding such an active compound for the treatment of individuals.

Toxicity and therapeutic efficacy of the ogerin composition of the present application can be determined by standard pharmaceutical procedures in experimental animals. While compounds that exhibit toxic side effects may be used, care should be taken to design a delivery system that targets such compounds to the site of affected tissue to minimize potential damage to uninfected cells and, thereby, reduce side effects.

The data obtained from animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to determine useful doses more accurately in humans. The pharmaceutical compositions can be included in a container, pack, or dispenser together with instructions for administration.

### IV. Kits

Another aspect of the application relates to a kit for prevention or treating post-operative abdominal adhesions. The kit comprises (1) an ogerin-comprising pharmaceutical composition formulated for intra-peritoneal injection or direct application to the peritoneal cavity; and (2) instructions for using the kit. In some embodiments, the kit further comprises a device for intra-peritoneal injection or direct application of the ogerin-comprising pharmaceutical composition to the peritoneal cavity.

The present application is further illustrated by the following examples that should not be construed as limiting. The contents of all references, patents, and published patent applications cited throughout this application, as well as the Figures and Tables, are incorporated herein by reference.

The present application is further illustrated by the following examples that should not be construed as limiting. The contents of all references, patents, and published patent applications cited throughout this application, as well as the Figures and Tables, are incorporated herein by reference.

### EXAMPLES

### Methods

Etiology of adhesion formation: Abdominal adhesion tissue is composed of a disorganized extracellular matrix (ECM) that connects the parietal peritoneum with intra-abdominal organs, or forms connections between organs, and is recalcitrant to normal tissue remodeling, resulting in impaired tissue function. The initial formation of adhesions is thought to be driven by exuberant fibrin clotting via macrophages in response to trauma or foreign body reactions to iatrogenic sutures or other surgical materials (such as surgical staples or any other material intentionally left behind after surgery), while adhesion maturation is largely driven by mesothelial cells and matrix-producing fibroblasts/myofibroblasts. Moreover, macrophages are a critical driver of sustained fibrosis in many tissues, in part via production of pro-fibrotic cytokines and modulation of ECM deposition by myofibroblasts. However, the inflammatory cell landscape during adhesion maturation has not been well characterized.

Murine Abdominal Adhesion Surgery: Mature adult (16-20-week-old) C57Bl/6J male and female mice underwent abdominal surgery to induce adhesions consistent with those observed postoperatively in clinical settings. Briefly, following sedation, a vertical midline laparotomy incision, extending from the xyphoid process to just above the bladder (˜4 cm), is made through the abdominal skin and musculature. The cecum is identified and eviscerated, followed by gentle abrasion with sterile 100-grit sandpaper for 30-60 seconds. Hemostasis is ensured and the cecum is then returned to the abdominal cavity. The abdominal wall is then abraded with sterile 100-grit sandpaper, followed by placement of two-four 4-0 silk sutures, which are placed into the abdominal wall. The intestines are then irrigated with warmed sterile saline, and hemostasis is confirmed. A small pinch of sterile autoclaved rice starch is then sprinkled on the cecum and abdominal sidewall. The laparotomy is closed with absorbable, interrupted sutures used to reapproximate the fascia and followed by sutures to close the skin. The skin is closed with skin adhesive.

Scoring of gross adhesion formation: At time of harvest, digital photographic images will be taken of the abdominal cavity of each mouse, with images standardized to a lateral reflection of abdominal wall prior to manipulation of intra-abdominal contents. These images are then curated in a Research Electronic Data Capture (RedCap)-based survey for blinded scoring. Adhesions are scored from 0-5: with ‘0’ indicating no adhesions; 1: a single thin, filmy adhesion; 2: more than one filmy adhesion; 3: one thick adhesion with focal attachment (+thin filmy adhesions); 4: one thick adhesion with broad planar attachment (+thin filmy adhesions); and 5: >1 thick adhesion with broad planar attachment, or >1 thick vascular adhesion with planar attachment (+thin filmy adhesions). Images are scored by at least 5 surgeons trained on the scoring process, with the mean of these scores reported for each mouse.

Histological assessment of adhesions and ileal smooth muscle hypertrophy: Following digital image capture, the area of adhesion between the abdominal wall and bowel (or other organs), including any bowel: bowel adhesions is isolated, fixed in 10% neutral buffered formalin, and processed for paraffin histology. In cases of adhesion absence, the abdominal wall and cecum are harvested and sectioned separately. Five-micron transverse sections are cut through the tissue. To ensure accurate quantification of adhesions, tissue sections are collected through multiple depths through the tissue. Hematoxylin & Eosin (H&E) staining are used to define overall tissue architecture and adhesion extent, while Masson's trichrome identify changes in extent of collagen deposition and degree of disorganization, indicative of a fibrotic response. Adhesion extent (length and cross-sectional area) along both the abdominal wall/cecal surface and between organs are quantified from H&E images by ≥2 blinded observers.

### Example 1

The study conducted a pilot, randomized, blinded trial of 20 mice.

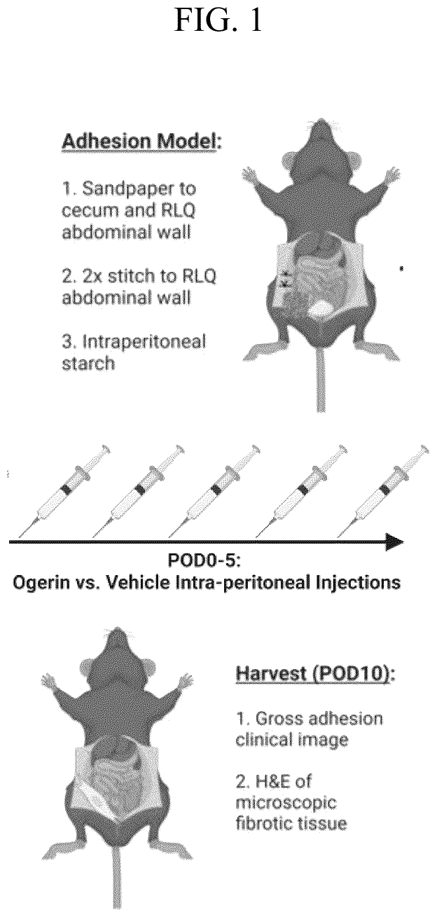

The study induced RLQ adhesions with a known model, taking sandpaper to the cecum and abdominal wall; placing 2 silk stitches, and applying peritoneal starch. Subsequently, the study injected ogerin or it's vehicle on post-operative day (POD) 0-5 into these mice. On POD10, the study harvested the mice for gross and molecular adhesion analysis; Vehicle, DMSO: Saline 60:40 (see FIG. 1).

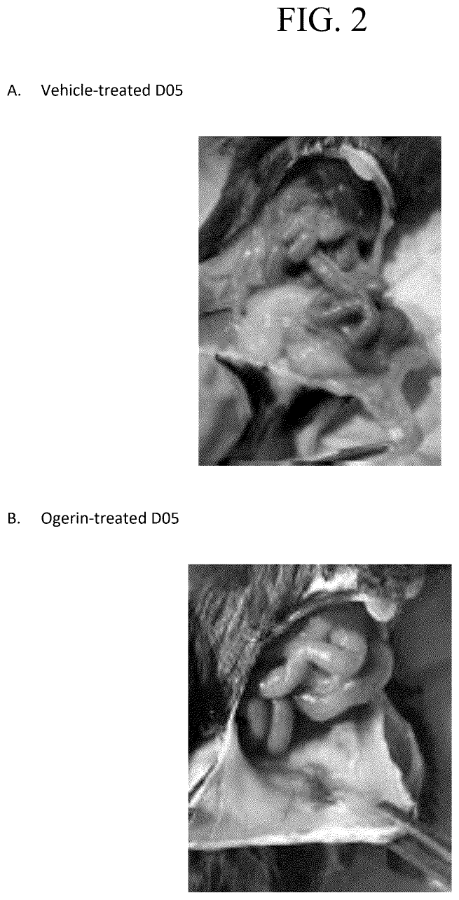

Remarkably, while all mice in the vehicle treated group formed robust adhesions (FIG. 2, Panel A), a substantial decrease in adhesion formation was observed in ogerin-treated mice, with 3 out of 4 ogerin-treated mice demonstrating a complete lack of adhesion formation (FIG. 2, Panel B). This work tests the ability of ogerin to prevent or blunt adhesion formation. These data identify a previously unknown application of ogerin.

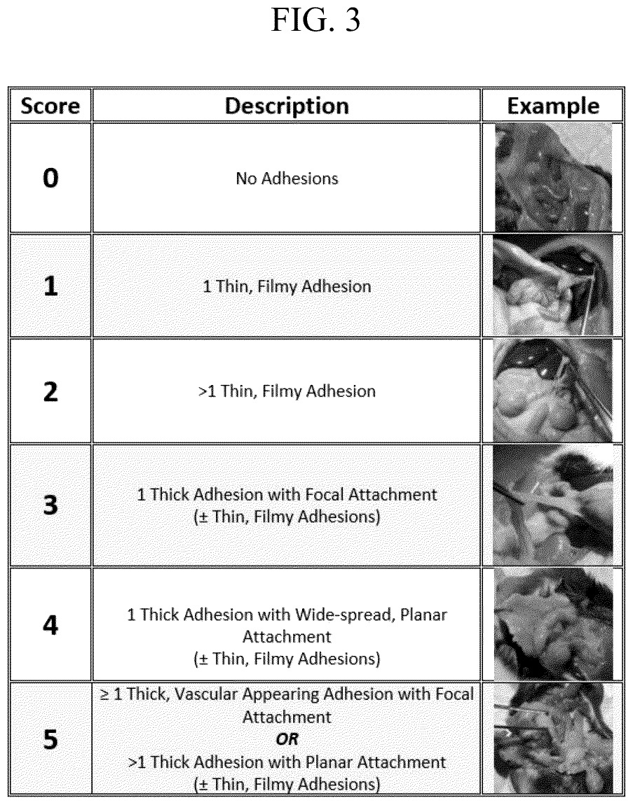

At harvest, the study took pictures of the peritoneal cavity, which were scored by 4 blinded, board certified surgeons adhesion scores ranged from 0 (none) to 5 (dense, vascular, adhesions). See FIG. 3.

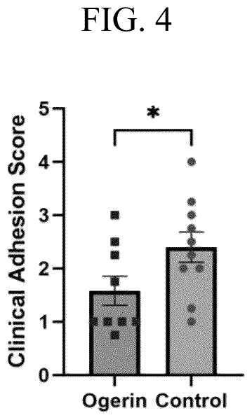

The study chose the area of highest visible fibrosis and adhesion density. This was typically near the site of the silk stitch in the abdominal wall. Adhesions were then measured in micrometers. Length of adhesions between the bowel to abdominal wall and bowel to bowel were measured. The total adhesion density (excluding abdominal wall fibrosis) was then measured by cross section. This is a good example of adhesion burden-hypothetically this cross section represents what general and pediatric surgeons may see in a single band obstruction event. As shown in FIG. 4, ogerin treated mice had significantly lower clinical adhesion scores.

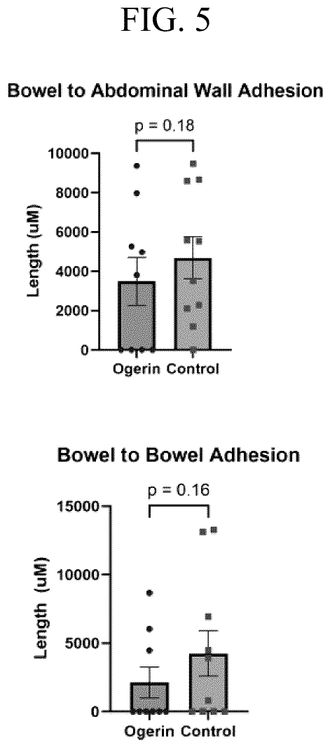

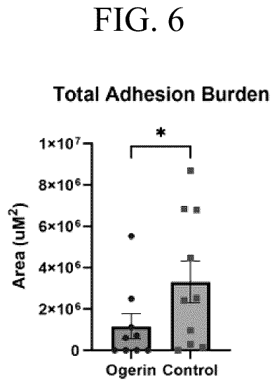

After measurement of all mice, the study found a reduction in length of adhesions between the bowel and abdominal wall and bowel to bowel. (See FIG. 5). The overall adhesion cross section was significantly lower in ogerin treated mice, compared to control (see FIG. 6).

The present application leveraged a murine model of abdominal adhesion formation that can be used to test the efficacy of different drugs to blunt or prevent adhesion formation. Down-regulation of the proton-sensing G-protein coupled receptor (GPCR), Gpr68/Ogr1, is a hallmark of idiopathic pulmonary fibrosis, a pathology driven by exuberant myofibroblast ECM production. Small molecule activation of Gpr68, via ogerin treatment, blunts TGF-b-induced myofibroblast differentiation in lung fibroblasts, and can promote reversion to a basal fibroblast phenotype following TGF-β-induced myofibroblast differentiation. Ogerin treatment is described herein to modulate both myofibroblast differentiation and reversion. Therefore, in herein, mice were treated with ogerin from DO-5 post-surgery to determine whether ogerin could blunt adhesion formation.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives there intended, unless the context specifically indicates the contrary.

What is claimed is:

1. A method of preventing or reducing development of post-surgical abdominal adhesions in a subject, comprising the steps of:
administering to the subject an effective amount of ogerin prior to an abdominal surgery or within 48 hours of the abdominal surgery.

2. The method of claim 1, wherein the subject is a human subject.

3. The method of claim 1, wherein the ogerin is administered within 24 hours of the completion of the abdominal surgery.

4. The method of claim 1, wherein the ogerin is administered within 12 hours of the completion of the abdominal surgery.

5. The method of claim 1, wherein the ogerin is administered via intra-peritoneal injection or direct application to the peritoneal cavity.

6. The method of claim 1, wherein the ogerin is administered at a dosage in the range of 0.1-40 mg/kg.

7. The method of claim 1, further comprising the additional step of:
applying a physical barrier to the surgery's site, wherein the physical barrier covers peritoneal surfaces until re-epithelialization is complete.

8. The method of claim 1, wherein the ogerin is administered at a dosage in the range of 0.1-20 mg/kg.

9. The method of claim 1, wherein the ogerin is administered within 12 hours prior to the abdominal surgery.

10. The method of claim 1, wherein the ogerin is administered to a subject who is asymptomatic for a presence of abdominal adhesions.

11. A method of treating post-surgical abdominal adhesion in a subject, comprising the step of:
administering to a subject in need of such treatment an effective amount of ogerin, wherein the subject has undergone abdominal or pelvic surgery and is at risk of post-surgical abdominal adhesion formation.

12. The method of claim 11, wherein the subject has undergone abdominal surgery and has one or more symptoms of post-surgical abdominal adhesions.

13. The method of claim 11, wherein the subject is a human subject.

14. The method of claim 11, wherein the ogerin is administered via intra-peritoneal injection or direct application to the peritoneal cavity.

15. The method of claim 11, wherein the ogerin is administered at a dosage of in the range of 0.1-40 mg/kg.

16. The method of claim 1, wherein the ogerin is administered at a dosage in the range of 0.1-20 mg/kg.
